(19) United States
(12) Patent Application Publication
(10) Pub. No.: US 2020/0368260 A1
(43) Pub. Date: Nov. 26, 2020

(54) USE OF BENZOPYRAN COMPOUND IN PREPARATION OF PRODUCT FOR REGULATING LIPID METABOLISM AND COMPOSITION OF THE SAME

(71) Applicant: Northwest Institute of Plateau Biology, Chinese Academy of Sciences, Xining City (CN)

(72) Inventors: Chenxu Ding, Xining City (CN); Kai Deng, Xining City (CN); Jing Meng, Xining City (CN); Na Hu, Xining City (CN)

(73) Assignee: Northwest Institute of Plateau Biology, Chinese Academy of Sciences, Xining City (CN)

(21) Appl. No.: 16/730,717

(22) Filed: Dec. 30, 2019

(30) Foreign Application Priority Data
May 23, 2019 (CN) 201910435112.2

(51) Int. Cl. A61K 31/7048; A61P 1/16

(52) U.S. Cl. CPC A61K 31/7048; A61P 1/16

(57) ABSTRACT

The present invention discloses the use of a benzopyran compound in the preparation of a product for regulating lipid metabolism, and a composition of the same, where the benzopyran compound includes petunidin-3-O-β-D glucoside and/or malvidin-3-O-β-D glucoside. It provides a new research direction for the preparation of the product for regulating lipid metabolism.

### TECHNICAL FIELD

The present invention relates to the technical field of medicine, and in particular to the use of a benzopyran compound in the preparation of a product for regulating lipid metabolism, and a composition of the same.

### BACKGROUND

Lipid metabolism is an important and complex biochemical reaction in vivo, which refers to a process in which the fat in a living organism is subjected to digestion and absorption, synthesis and decomposition with the help of various related enzymes, and processed into substances needed by the body to ensure the operation of normal physiological functions, which is of great significance to life activities. The lipid is an important substance for energy storage and energy supply of the body, and is also an important structural ingredient of a biofilm. Diseases caused by abnormal lipid metabolism are common diseases in the modern society.

Lipid deposition refers to the occurrence or significant increase of lipid droplets in parenchyma cells other than fat cells, and fatty degeneration is more common in infection, alcoholism, hypoxia, poisoning, diabetes, obesity and malnutrition. Fatty degeneration is mainly found in parenchymatous organs such as the liver, heart, kidney, skeletal muscle and the like.

Nonalcoholic fatty liver disease (NAFLD) is a metabolic stress-induced liver injury closely related to insulin resistance (IR) and genetic susceptibility, its pathological changes are similar to those of the alcoholic liver disease (ALD), but the patient has no history of excessive drinking, and its disease spectrum includes nonalcoholic simple fatty liver (NAFL), nonalcoholic steatohepatitis (NASH) and their related liver cirrhosis and hepatocellular carcinoma.

In clinical diagnosis, the definitive diagnosis of NAFLD should meet the following 3 conditions: (1) having no drinking history or drinking converted amount of ethanol being <140 g/week (female <70 g/week); (2) specific diseases which can cause fatty liver, such as viral hepatitis, drug-induced liver diseases, total parenteral nutrition, hepatolenticular degeneration, autoimmune liver diseases, etc., being excluded; and (3) histological changes in liver biopsy meeting the pathological diagnostic criteria for fatty liver diseases. An important task in the treatment of NAFLD is to reduce fat deposition in the liver and avoid NASH and hepatic dysfunction due to “second strike”, and a NASH patient needs the prevention of liver disease progression, reduction or prevention of the occurrence of liver cirrhosis, liver cancer and their complications. At present, the main measures for the treatment of the nonalcoholic fatty liver disease are: exercising, improving the diet structure, controlling the body weight, reducing the waist circumference; improving IR, and correcting metabolic disorders, where an insulin sensitizer can be used. For a liver-protection and anti-inflammatory drug, the function and status of such a drug in the prevention and treatment of NAFLD are still controversial. There is currently no sufficient evidence to recommend the routine use of such a drug in patients with NAFLD/NASH (“Guidelines for the diagnosis and treatment of nonalcoholic fatty liver diseases” in 2010), so now it is urgently needed to develop a natural treatment for the NAFLD.

At present, no related report of using the benzopyrazole compound such as petunidin-3-O-β-D glucoside and/or malvidin-3-O-β-D glucoside for regulating lipid metabolism has been seen.

### SUMMARY

An objective of the present invention is to provide the use of a benzopyran compound in the preparation of a product for regulating lipid metabolism, and a composition of the same, where the benzopyran compound such as petunidin-3-O-β-D glucoside and/or malvidin-3-O-β-D glucoside, is used for the relief and treatment of diseases related to obesity, nonalcoholic fatty liver and lipid metabolism disorders, and the benzopyran compound is further used as an active ingredient of the composition to obtain a good therapeutic effect, which provides a new research direction for preparing a product for regulating lipid metabolism.

The present invention provides the use of a benzopyran compound in the preparation of a product for regulating lipid metabolism, where the benzopyran compound includes petunidin-3-O-β-D glucoside and/or malvidin-3-O-β-D glucoside, which have the structural formulas as follows:

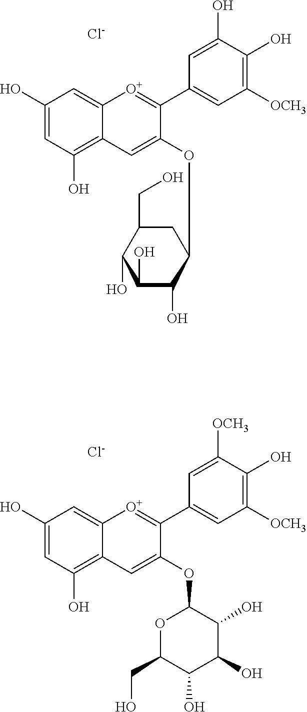

petunidin-3-O-β-D glucoside malvidin-3-O-β-D glucoside

In the use provided by the present invention, the product may be a drug, a health care product, an experimental agent, or other products that modulate lipid metabolism. When it is a drug, the drug is used for the prevention and/or treatment of one or more of diseases associated with obesity, fatty liver, and lipid metabolism disorders.

The lipid metabolism disorder refers to the abnormality in the quality and quantity of lipids (lipins) and their metabolites in blood and other tissues and organs caused by congenital or acquired factors.

Further, the product is a lipid deposition inhibitor.

The lipid deposition inhibitor is a product that promotes metabolism of fat cells, and this product can prevent fat from being accumulated in organs and tissues such as liver, brain, kidney, etc. due to slow metabolism.

Further, the product is an autophagy modifier.

Autophagy is a process in which a cell phagocytizes its own cytoplasmic proteins or organelles and enables the same to be coated into a vesicle, the vesicle is fused with a lysosome to form an autolysosome, and the contents encapsulated by the autolysosome are degraded, thereby realizing the metabolism needs of the cell itself. Autophagy plays an important role in the process of lipid metabolism. The autophagy modifier is a product which achieves the aim of regulating lipid metabolism by adjusting cell autophagy.

Further, the product is an autophagy inducing agent.

The autophagy inducing agent is a product which induces an adipocyte to enhance the autophagy activity of the adipocyte.

Further, the product is one or more of a TC inhibitor, a TG inhibitor, an ALT inhibitor, and an AST inhibitor.

The TC, TG, ALT or AST inhibitor, is a product which reduces the level of TC, TG, ALT or AST by promoting the metabolism actions of liver lipid cells.

Further, the nonalcoholic fatty liver is nonalcoholic simple fatty liver or/and nonalcoholic steatohepatitis.

Further, the product is a cell agonist, and preferably a nonalcoholic fatty liver cell agonist.

ROS is an oxygen free radical in a cell. Excessive ROS will damage mitochondria and other organelles of the cell, and thus the ATP reduction of the cell may make it impossible for the cell to continue the completion of a normal lipid metabolism reaction.

The cell is a product capable of enhancing the activity of another cell and promoting the metabolic reaction of adipocytes, such as a pharmaceutical formulation, and the nonalcoholic fatty liver cell agonist is a product that enhances the activity of the nonalcoholic fatty liver cell and promotes the cellular metabolism reaction of fatty liver cells.

Further, the nonalcoholic fatty liver is nonalcoholic simple fatty liver or/and nonalcoholic steatohepatitis.

The present invention provides a composition for regulating lipid metabolism based on the research of the use of the benzopyran compound, where the composition includes a combination of one or more of the benzopyran compound or a stereoisomer, hydrate, solvate, pharmaceutically acceptable salt or eutectic thereof.

A composition of a benzopyran compound for regulating lipid metabolism, includes active ingredients of petunidin-3-O-β-D glucoside and/or malvidin-3-O-β-D glucoside.

The active ingredient of the present invention refers to a part that exerts a therapeutic effect in the product.

The beneficial effects of the present invention are: in contrast of the prior art,

1. the inventor uses a MTT cell viability experiment to qualitatively study the contents of TG, TC, ALT and AST in a cell of a nonalcoholic fatty liver disease model, the experimental results show that when a HepG2 cell is treated with anthocyanin and petunidin-3-O-β-D glucoside, and malvidin-3-O-β-D glucoside for 6 h, the cell viability is significantly increased, and the treatment of the cell with the anthocyanin and petunidin-3-O-β-D glucoside, and malvidin-3-O-β-D glucoside has no cytotoxicity over time;

2. it can be known from the experimental study that, petunidin-3-O-β-D glucoside and malvidin-3-O-β-D glucoside can reduce the levels of TC, TG, ALT and AST in a high fat cell, as compared with a control group and a low dose group, the treatment groups with medium and high doses have a very significant reduction; and

3. the inventor studies the effect of petunidin-3-O-β-D glucoside and malvidin-3-O-β-D 5 glucoside on autophagy by Western blot and a Q-PCR experiment, including Bcl-1, p62, Apg5L, APG7 and Fas, the results of Western blotting show that the low doses of petunidin-3-O-β-D glucoside and malvidin-3-O-β-D glucoside have a significant effect on the protein expression of APG5L, APG7, p62 and Bcl-1, in the specific experimental results, a inhibition trend is shown in the high dose group, the results of Q-PCR show that, petunidin-3-O-β-D glucoside and malvidin-3-O-β-D glucoside can significantly decrease the mRNA expression level of Bcl-1 and Fas, and up-regulate the level of APGSL.

### DESCRIPTION OF THE DRAWINGS

FIG. 1 is a graph showing the effect of a concentration of Lycium Ruthenicum anthocyanin on the viability of a HepG2 cell according to the present invention;

FIG. 2 is a graph showing the effect of anthocyanin on the cellular reactive oxygen according to the present invention;

FIG. 3 is a graph showing the effect of the time of the treatment with petunidin-3-O-β-D glucoside and malvidin-3-O-β-D glucoside on the cell viability;

FIGS. 4A-4D are graphs showing the effect of petunidin on the TC, TG, ALT and AST of a NAFLD cell, respectively;

FIGS. 5A-5D are graphs showing the effect of malvidin on the TC, TG, ALT and AST of a cell, respectively;

FIGS. 6A-6G-2 are graphs showing the effect of petunidin-3-O-β-D glucoside (Pt) and malvidin-3-O-β-D glucoside (Mv) on related protein expression and mRNA expression in oleic acid induced HepG2 cells;

FIG. 6A shows the result of Pt western blotting experiment.

FIG. 6B shows the result of Mv western blotting experiment.

FIGS. 6C-1 to 6C-4 show the column bar analysis of Pt western blotting experiment.

FIGS. 6D-1 to 6D-4 show the column bar analysis of Mv western blotting experiment.

FIGS. 6E-1 to 6E-4 show the result of Pt QPCR experiment on APGSL, Apg7, p62, and Bcl-1, respectively.

FIGS. 6F-1 to 6F-4 show the result of Mv QPCR experiment on APGSL Apg7, p62, and Bcl-1, respectively.

FIGS. 6G-1 to 6G-2 show the result of Pt and Mv QPCR experiment on Fas. **P<0.01,*P<0.05, anthocyanins treated vs Control group; Blk, normal HepG2 cells; Ctl, anthocyanins untreated groups.

### DETAILED DESCRIPTION

According to the relevant literature search, the results showed that there was a link between the lipid metabolism disorder and autophagy, petunidin-3-O-β-D glucoside and malvidin-3-O-β-D glucoside had good antioxidant activities, and the anthocyanin extract had a regulatory effect on lipid metabolism. We first studied the effects of petunidin-3-O-β-D glucoside, malvidin-3-O-β-D glucoside and Lycium Ruthenicum anthocyanin on the cell viability; and meanwhile based on a high-fat HepG2 cell model obtained by oleic acid treatment at the early stage, then we selected LC-MS to qualitatively analyze the mother nuclei of petunidin-3-O-β-D glucoside and malvidin-3-O-β-D glucoside, and detected the total triglyceride, total cholesterol, glutamic pyruvic transaminase and glutamic oxaloacetic transaminase of the cells after the high-fat HepG2 cell model was treated for 6 h; and at the same time, the expression levels of FAS and autophagy related pathways were detected at a protein level by means of Western blotting technology; and the expression in a mRNA level of autophagy related gene was detected by a fluorescence quantitative PCR technology.

1.1 Experimental Materials, Reagents and Instruments

1.1.1 Sources of Test Samples

The test samples included 2 anthocyan compound monomers (purchased from Sigma): Pt (petunidin-3-O-β-D glucoside), Mv (malvidind-3-O-β-D glucoside), as well as an anthocyanin crude extract LRA prepared in the laboratory, where the chemical structural formulas of petunidin-3-O-β-D glucoside and malvidin-3-O-β-D glucoside were as follows:

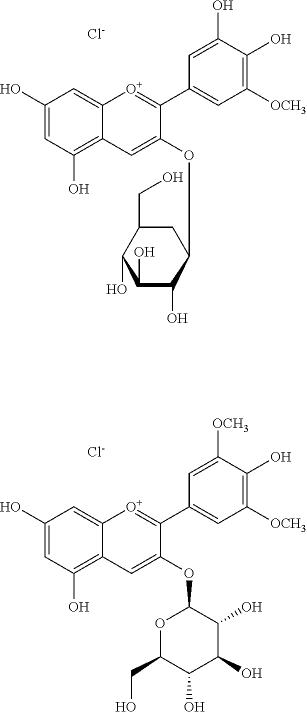

petunidin-3-O-β-D glucoside malvidin-3-O-β-D glucoside

1.1.2 Materials and Reagents

In this application, the HepG2 human hepatoma cell line was provided by the Shanghai Cell Bank of the Chinese Academy of Sciences, and all of the primers for genes such as APGSL, Apg7, p62, Bcl-1, and FAS were synthesized by Sangon Biotech (Shanghai) Co., Ltd.

Formulation of Main Reagents

The MTT solution required for this experiment and the western blotting related reagents were formulated as follows:

(1) Formulation of MTT Solution

25.0 mg of MTT powder was weighed and fully dissolved in 5 mL of PBS to formulate a solution with a concentration of 5 mg/mL, sub-packaged and then stored with protection from light at −20° C.

(2) Formulation of Oil Red O Dye Liquor

Formulation of oil red O mother liquor: 0.60 g of oil red O powder was weighed into 100 mL of isopropanol, stirred well with a magnetic stirrer, shaken uniformly, and filtered with filter paper to prepare an oil red O stock solution.

Formulation of oil red O working solution: the above oil red O stock solution was taken, fully mixed with deionized water according to a proportion of 3:2, and then filtered to formulate an oil red O working solution, which was then used within 2 hours.

(3) Formulation of Neutral Formaldehyde Solution

10 mL of formaldehyde (40%), 0.40 g of sodium dihydrogen phosphate, and 0.65 g anhydrous disodium hydrogen phosphate were measured, added with 90 mL of deionized water, and mixed for dissolution.

(4) Electrophoresis Buffer (5×)

7.58 g of Tris, 46.90 g of Glycine, and 2.50 g of SDS were accurately weighed, added with a small amount of deionized water, continually stirred for complete dissolution, then brought to a constant volume of 500 mL, and stored at room temperature.

Electrophoresis buffer (1×): 20 mL of an electrophoresis buffer (5×) was measured and added with 480 mL of deionized water to formulate a 1× electrophoresis buffer.

(5) Transfer Buffer (10×)

15.15 g of Tris and 72.00 g of Glycine were accurately weighed, added with a small amount of deionized water, continually stirred for complete dissolution, then brought to a constant volume of 500 mL by using a volumetric flask, and stored at room temperature.

Transfer buffer (1×): 70 mL of a transfer buffer (10×) was measured by a measuring cylinder, and then added with 140 mL of methanol and 490 mL of deionized water (according to the proportion of transfer buffer (10×):methanol:deionized water =1:2:7) to formulate into the 1× transfer buffer.

(6) TBS (10×)

12.12 g of Tris and 40.04 g of NaCl were accurately weighed, added with a small amount of water until they were completely dissolved, brought to a constant volume of 500 mL, determined for a pH value with a pH meter and adjusted to pH 7.5 with concentrated hydrochloric acid, and stored at room temperature.

TBST (1×): a 1× TBST was formulated according to the proportion of deionized water:Tween 20=1:9:1%.

(7) 10% Ammonium Persulfate (AP)

40.00 mg of AP was accurately weighed, added with 400 μL of deionized water, shaken by overturning until it was completely dissolved, and stored at 4° C. for later use.

(8) 5% Blocking Solution (BSA)

1.00 g of BSA or skimmed milk powder was accurately weighed, added with 20 mL of a 1×TBST solution, shaken by overturning until it was completely dissolved, and stored at 4° C. for later use.

(9) 5× SDS Loading Buffer

2.5 mL of 0.5 mol/L Tris-HCl (pH 6.8), 0.39 g of dithiothreitol (DTT), 0.50 g of SDS, 2.5 mL of glycerin, and 25.0 mg of bromophenol blue were accurately weighed, continually stirred until they are fully mixed, sub-packaged in a 1.5 mL sterile centrifuge tube, and stored at 4° C.

A. Formulation of DNase

30 μl of a DNA enzyme was added with 40 μl of RDD and 10 μl of DEPC water, where the formulation of a DW washing liquor: DW:anhydrous ethanol0=9:1

1.2 Experimental Method

1.2.1 Effect of a Concentration of Lycium Ruthenicum Anthocyanin on the Proliferation Viability of HepG2 Cells

Lycium Ruthenicum anthocyanin was taken and added with DMEM high-glucose medium to formulate into 0, 5, 10, 20, 40, 80, 160, 320, 640, and 1280 μg/mL, and then used for treating the HepG2 cells for 6 h. The specific steps of an MTT cell proliferation viability experiment were as follows:

(1) Thawing and Cryopreservation of the HepG2 Cell

Cryopreservation of HepG2 cells: when the cells were grown to a confluence of 80% in a culture dish, the cells were digested with 0.25% EDTA-trypsin, the digested cell suspension was pipetted into a centrifuge tube and centrifuged, the supernatant was discarded, the pellet was added with a cell cryopreservation solution (DMEM high-glucose medium:FBS:DMSO=5:4:1) was gently pipetted up and down to uniform, then the cells were pipetted into a cryo tube, slowly frozen in a refrigerator at 4° C. for 10 min, and then placed into a refrigerator of −20° C. to continue the frozen for about 1 h, and finally the cryo tube was placed at −80° C. overnight, and then placed into a liquid nitrogen tank of −198° C. on the next day for long-term cryopreservation.

Thawing of Hepg2 cells: The cryo tube of HepG2 cells was removed from the liquid nitrogen tank, rapidly placed into a 37° C. water bath for melting, and then the cells were transferred to 10 mL of a high-glucose medium containing 10% FBS, incubated under 5% CO2 and saturated humidity at 37° C. with the culture medium being replaced every two days, and subjected to passage when the cells grown to a confluence of 80%.

(2) Culture and Passage of HepG2 Cells

Culture of HepG2 cells: the HepG2 cells were placed into a high-glucose culture medium containing 10% FBS and 1% penicillin-streptomycin, incubated under 5% CO₂ and saturated humidity at 37° C. with the culture medium being replaced every two days, and subjected to passage when the cells grown to a confluence of 80%.

Passage of HepG2 cells: When the cell confluence reached about 80%, the cells were subjected to passage. During passage, first the culture medium was pipetted off, and the cells were washed with an appropriate amount of PBS for 2 times, and added with 1 mL of a trypsin digestion solution, the culture dish was gently shaken such that the trypsin digestion solution was evenly distributed in the culture dish, the cells were digested at 37° C. for about 2 min until the cells were substantially detached from the bottom wall of the culture dish, the culture dish was gently shaken and added with an appropriate amount of a DMEM culture medium, and the cells were pipetted up and down repeatedly to completely detach the cells from the bottom of the culture dish and disperse into individual cells. The cell suspension was transferred to a new culture dish in an appropriate proportion, then added with a fresh DMEM high-glucose culture medium and 10% FBS, and then cultured in a CO2 constant-temperature incubator.

(3) Counting of HepG2 Cells

A hemocytometer having an H-shaped groove and a strut on each side of the groove, was covered by a cover glass to form a 0.1 mm counting cell. When the experiment required a certain density of the cells, the cells could be diluted by a certain multiple, the cell suspension was shaken well and pipetted by a pipette gun at a small amount, a small drop of it was dropped from a lower edge of a middle platform of a technical plate so that the counting area was filled full of the cell suspension without allowance of bubble generation in this process, then the counting plate was placed under a microscope to record the total number of cells in 4 large squares, and finally the cell number was calculated according to the following formula:

cell number=the total number of cells in 4 squares/4×10⁴×a dilution multiple

The cell density of the suspension was determined by the number of cells, and the cell density was adjusted according to the requirements of the experiment. Effect of oleic acid on the proliferation viability of HepG2 cells

The HepG2 cells were seeded into a 96-well culture plate at 1×10⁴ cells per well, incubated in a DMEM culture medium containing 10% fetal bovine serum for 12 h until complete confluence of the cells, and then the culture medium was replaced by respective concentrations of 0, 0.5, 1, 1.5, 2, 2.5, 3, 3.5, 4 mM of oleic acid formulated with a serum-free culture medium to treat the cells for 0, 24, 48, and 72 hours, with 4 replicate wells being set for each concentration. After the completion of the incubation, 10 μL of a freshly prepared MTT solution at a final concentration of 5 mg/mL was added into each well and the culture was continued for 4 h. Then, the liquid in the culture well was pipetted off to terminate the reaction. Each well was added with 100 μL of DMSO, and shaken fully on a plate shaker for 10 mM such that the crystalline was fully dissolved, and an A490 absorbance was measured on a microplate reader. The cell proliferation viability was calculated according to the following formula.

Cell proliferation viability (%)=(the OD of drug group/the OD of blank group)×100%

When the cell proliferation viability was greater than 80%, it was considered that the drug has little effect on the cell viability, i.e., exhibiting no toxic effects, and this was used as the standard for selecting the optimal concentration of the drug in this study, and is used for determining the optimal range for the working concentration of a further experiment.

1.2.2 Effect of Lycium Ruthenicum Anthocyanin on the Proliferative Viability of HepG2 Cells

Detection of reactive oxygen levels in HepG2 cells by fluorescence microscopy

The culture, induction and drug treatment of the cells were the same as those in 1.2.1. The cell treatment manner was carried out according to the operation instructions of the kit. The fluorescence intensity was observed in a FITC channel of a fluorescence microscope and photographed.

1.2.3 Inhibition Effects of petunidin-3-O-β-D glucoside and malvidin-3-O-β-D glucoside on Cell Lipid D5eposition

1. The culture, model establishment and experimental steps of the cells were operated according to 1.2.1. When the cells were cultured to the confluence of 70%, the cells were treated with 0.5 mM for 24 h to establish a cell model of nonalcoholic fatty liver disease. The anthocyanin compound mother liquor was diluted to 10 μM to treat the cells for 0, 6, 12, 24, and 48 h. The MTT cell proliferation viability experiment determined the optimal dosing time.

2. The culture, model establishment and experimental steps of the cells were operated according to the above steps. The anthocyanin compound mother liquor was diluted to 0, 0.1, 1, 10, 50, and 100 μM to treat the high-fat HepG2 cells, respectively.

3. The culture solution was pipetted off, gently washed with PBS for 3 times, and added with trypsin to digest the cells for 1 min The cell suspension was pipetted into a 1.5 mL sterile EP tube, centrifuged at 1000 rpm for 10 minutes, the supernatant was discarded, the pellet was added with an appropriate amount of PBS, ultrasonically crushed under the condition of an ice-water bath, and the prepared homogenate was not centrifuged and was ready for determination.

4. Detection of the Triglyceride (TG) Content of a Cell

The intracellular triglyceride content was determined according to the operating instructions of a triglyceride (TG) kit. The protein content in the cells was determined by a BCA protein concentration kit, and the triglyceride content in the cells was calibrated with the protein content.

5. Detection of Total Cholesterol (TC) Content in the Cells

The total cholesterol (TC) content in the cells was determined according to the operating instructions of a TC kit. The protein content in the cells was determined by a BCA protein concentration kit, and the total cholesterol content in the cells was calibrated with the protein content.

6. Detection of Cellular Alanine Aminotransferase (ALT) Content

The intracellular alanine aminotransferase (ALT) content was determined according to the operating instructions of an ALT kit.

7. Detection of Cellular Aspartate Aminotransferase (AST) Content

The intracellular aspartate aminotransferase (AST) content was determined according to the operating instructions of an AST kit. 1.2.4 Effect of petunidin-3-O-β-D glucoside and malvidin-3-O-β-D glucose on autophagy of HepG2 cells

The culture, model establishment and drug treatment manners of the cells were operated according to 1.2.3. After completion, cells were collected, cytoplasmic proteins were extracted, and protein electrophoresis was conducted after quantification. Particularly the following methods were used:

When the cell confluence reached 70%, 0.5 mM oleic acid was added to treat the cells for 24 h. After completion, the cells were collected and the cytoplasmic protein was extracted, and the protein electrophoresis was conducted after quantification. The electrophoresis method was as follows:

1. Extraction of Total Cellular Protein

After cell culture was completed, the culture plate was taken out, the culture medium in the well was discarded, the pellet was washed twice with PBS, placed onto an ice bath, and added with 100 μL of a protein lysis buffer (RIPA) (PMSF was added into the cell lysis buffer at 1:1000 within few minutes before the use, with a final concentration of 1 mM) per well. After the cells were lysed for about 10 mM, the cells were scraped off with a cell scraper, pipetted into a 1.5 mL centrifuge tube (which could be crushed with an ultrasonic cell disruptor for 3-5 times), then centrifuged at 12,000 rpm under 4° C. for 15-20 mM. The supernatant was taken and transferred into a new centrifuge tube for protein quantification or storage at −80° C.

2. Determination of Protein Concentration

Theprotein content was determined by a BCA method, and the operation method was carried out according to the instructions of the kit. After the protein concentration was measured, the protein sample was added with a 5× loading buffer and mixed uniformly at 1:4, then subjected to water bath at 100° C. for 10 mM (entangling with a sealing film to prevent bouncing off), and stored at 4° C.

3. SDS-PAGE Electrophoresis

The specific operation method of protein electrophoresis was in accordance with the relevant references. Since each of the molecular weight of the target proteins was within 10-80 KDa except for FASN (the separation gel was prepared according to the following table), the separation gel concentration was selected to be 10%, and the concentration of the FASN protein separation gel was selected to be 6%.

Formulation Table of 6% SDS-PAGE Separation Gel

| Components | Different volumes of 6% SDS-PAGE separation gel (mL) | | | | |
|---|---|---|---|---|---|
| (mL) | 5 | 10 | 15 | 20 | 30 |
| Distilled | 2.6 | 5.3 | 7.9 | 10.6 | 15.9 |
| Water | | | | | |
| 30% | 1.0 | 2.0 | 3.0 | 4.0 | 6.0 |
| Acrylamide | | | | | |
| 1.5M Tris- | 1.3 | 2.5 | 3.8 | 5.0 | 7.5 |
| HC1 pH 8.8 | | | | | |
| 10% SDS | 0.05 | 0.1 | 0.15 | 0.2 | 0.3 |
| 10% AP | 0.05 | 0.1 | 0.15 | 0.2 | 0.3 |
| TEMED | 0.004 | 0.008 | 0.012 | 0.016 | 0.024 |

The optimal separation ranges for SDS-PAGE separation gels of different concentrations were as follows:

| Concentration of acrylamide (%) | The optimal separation range (KDa) |
|---|---|
| 15 | 10-40 |
| 12 | 12-60 |
| 10 | 20-80 |
| 8 | 30-90 |
| 6 | 50-150 |

The separation gel was formulated according to the following table. Formulation table of 10% SDS-PAGE separation gel

| Components | Different volumes of 10% SDS-PAGE separation gel (mL) | | | | |
|---|---|---|---|---|---|
| (mL) | 5 | 10 | 15 | 20 | 30 |
| Distilled | 1.9 | 4.0 | 5.9 | 7.9 | 11.9 |
| Water | | | | | |
| 30% | 1.7 | 3.3 | 5.0 | 6.7 | 10 |
| Acrylamide | | | | | |
| 1.5M Tris- | 1.3 | 2.5 | 3.8 | 5.0 | 7.5 |
| HCl pH 8.8 | | | | | |
| 10% SDS | 0.05 | 0.1 | 0.15 | 0.2 | 0.3 |
| 10% AP | 0.05 | 0.1 | 0.15 | 0.2 | 0.3 |
| TEMED | 0.002 | 0.004 | 0.006 | 0.008 | 0.012 |

A spacer gel was formulated according to the following table, and sample loading was conducted. After completion, electrophoresis should be conducted as soon as possible, where concentration was conducted at 80 v for 30 min, and then electrophoresis was conducted at 120 v until the front edge ran to the lower edge of the separation gel, and the electrophoresis was stopped.

Formulation Table of 5% SDS-PAGE Spacer Gel

| Components | Different volumes of 10% SDS-PAGE separation gel (mL) | | | | | |
|---|---|---|---|---|---|---|
| (mL) | 2 | 3 | 4 | 5 | 6 | 8 |
| Distilled | 1.4 | 2.1 | 2.7 | 3.4 | 4.1 | 5.5 |
| Water | | | | | | |
| 30% | 0.33 | 0.5 | 0.67 | 0.83 | 1.0 | 1.3 |
| Acrylamide | | | | | | |
| 1M Tris-HC1 | 0.25 | 0.38 | 0.5 | 0.63 | 0.75 | 1.0 |
| pH 6.8 | | | | | | |
| 10% SDS | 0.02 | 0.03 | 0.04 | 0.05 | 0.06 | 0.08 |
| 10% AP | 0.02 | 0.03 | 0.04 | 0.05 | 0.06 | 0.08 |
| TEMED | 0.002 | 0.003 | 0.004 | 0.005 | 0.006 | 0.008 |

3. Transferring The PVDF membrane was immersed in methanol for 1 min in advance and then soaked with a 1× transfer solution for 10 min. the gel was removed, the excess gel was cut off from top and bottom and left and right, and then the gel was assembled in the order of filter paper-gel-membrane-filter paper from a blackboard to a whiteboard, the bubbles were removed from therebetween, and the assembly was clamped by a clip, and placed in a transfer slot with the black side to black side, and white side to white side.

The transferring was generally conducted by a constant-current method, in which the transfer current and the transfer time were selected according to the molecular weight of the target protein.

4. Blocking

After the completion of the transfer, the PVDF membrane was blocked with a TB ST room-temperature shaker containing 5% skim milk powder for 1 hour.

5. Incubation with Antibodies

The primary antibody was diluted with TBST containing 5% BSA or 5% skim milk powder (see the antibody instructions for specific dilution multiple). After the end of blocking, the PVDF membrane was incubated with the well-diluted primary antibody overnight at 4° C. After the end of incubation, the membrane was washed for 5 times with TBST, 10 min for each time. The secondary antibody was diluted with TBST containing 5% skim milk powder (1:2000). After the completion of the incubation with the primary antibody, the membrane was incubated with a corresponding HRP-labeled secondary antibody for 1 h at room temperature, and washed for 5 times with TBST, 10 min for each time.

6. Developing

The membrane was detected by an ECL method, in which a liquid and a B liquid were mixed at 1:1 to formulate a developing solution, the developing solution is prepared immediately before use, and each membrane was added with about 100-200 μL of the developing solution.

1.2.5 Effects of petunidin-3-O-β-D glucoside and malvidin-3-O-β-D glucoside on the Transcription Level of Autophagy-Related Genes of HepG2 Cells

The culture, model establishment and drug treatment manners of the cells were the same as those in 1.2.4. After the completion of the treatment, the cells were collected, total RNA was extracted, then cDNA was synthesized by reverse transcription, and finally, the change of the transcription level of autophagy-related genes of the HepG2 cell in a nonalcoholic fatty liver disease model under the actions of petunidin-3-O-β-D glucoside and malvidin-3-O-β-D glucoside was detected by the real-time fluorescent quantitative PCR method, where the following method can be adopted:

(1) Extraction of Total Cellular RNA by Trizol Method

1. After the completion of drug treatment, the cells were washed with PBS for 2 times, added with 1 mL Trizol per about 1×10⁷ cells, then pipetted up and down with a 1 mL pipette gun (free of RNA enzymes) until the liquid was clear and had no cell aggregate, transferred into a 1.5 μL RNA-enzyme-free EP tube, mixed well by inverting up and down for 10 times, and lysed for 5 minutes at room temperature.

2. The cells were added with 0.2 mL of chloroform, shaken vigorously for 30 sec, and allowed to stand for 3 min at room temperature. Centrifugation was conducted at 12,000 rpm at 4° C. for 4 min.

3. The upper aqueous phase was pipetted and transferred into a clean centrifuge tube, added with ½ volume of absolute ethanol, and mixed well.

4. The adsorption column was placed into a collection tube, and the adsorption column was added with all of the solutions and translucent fibrous suspensions with a pipette, allowed to stand for 2 min, and centrifuged at 12,000 rpm for 3 min, and the waste fluid in the collection tube was discarded.

5. The adsorption column was placed back into the collection tube, added with 500 μL of an RPE solution, allowed to stand for 2 min, and centrifuged at 10,000 rpm for 30 sec, and the waste fluid in the collection tube was discarded. The step 4was repeated once more, and the adsorption column was placed back into the collection tube and centrifuged at 10,000 rpm for 2 min.

6. The adsorption column was placed into a 1.5 mL clean centrifuge tube, added with 30 μL of DEPC-treated ddH₂O at the center of the adsorption membrane, allowed to stand for 5 min, and centrifuged at 12,000 rpm for 2 min, and the resultant RNA solution was stored at −70° C.

(2) RNA Detection and Quantification

1. Determination of RNA Concentration and Purity

1 μL of an RNA mother liquor and 19 μL of DEPC water were taken, centrifuged and mixed well. 1 μL of the mixture was taken and transferred onto a 24-well elisa plate to determine A260, A280 and A230, and three values, the A260/A280, A260/A230, and the DNA concentration, were calculated. If the A260/A280 ratio was between 1.8-2.0, then it indicated that the RNA purity was good. The volume of the RNA mother liquor required for 1000 ng of RNA was calculated based on the finally determined concentration. The diluted sample was then subjected to agarose electrophoresis to detect whether the RNA is degraded or doped with impurities.

2. Identifying RNA Integrity By Agarose Electrophoresis

1) Preparation of a 1% agarose gel: 0.40 g agarose was added into 40 mL of a 1× TAE electrophoresis buffer (or DEPC water), heated in a microwave oven for 2 min so that the agarose was dissolved to completely clear, cooled to about 60° C., added with 2 μL of a 1 mg/mL ethidium bromide solution, and mixed well, the gel was poured into a gel preparation slot into which a comb had been inserted vertically, and the comb was pulled out after the gel was completely solidified. About 700 mL of the 1× TAE electrophoresis buffer formulated in advance was added to the electrophoresis tank, and the solidified agarose gel was placed into the electrophoresis tank and ready for sample loading.

2) Sample loading: 1 μL of the RNA mother liquor and 1 μL of a 6× DNA loading buffer were mixed well in a 200 μL RNA-enzyme-free EP tube by means of a pipette gun, and then added into a loading well with the pipette gun.

3) Electrophoresis: electrophoresis was conducted at 100 V for about 17 min.

4) Analysis: the gel that had subjected to the electrophoresis was placed into an UV imaging system for observation, and if the bands of 28S and 18S were clear, the brightness of the former was about 2 times larger than that of the latter, the band of 5S was weak, it indicated that the RNA integrity was good.

(3) Reverse Transcription

1. The first strand of cDNA was synthesized by using 800 ng of total RNA as a template and a total system of 20 μL. A nuclease-free PCR tube in the ice bath was added with the following reagents:

| Reaction System | Volume (μL) |
|---|---|
| RNA sample (with a volume of 800 ng) | X |
| Random Primer p(dN)₆ (100 pmol) | 1 |
| dNTP Mix (0.5 mM final concentration) | 1 |
| Rnase-free H₂O | Brought to a constant |
| | volume of 14.5 μL |

2. The reagents were mixed gently to uniform and centrifuged for 3-5 s, the reaction mixture was placed in a 65° C. warm bath for 5 min and then in an ice bath for 2 min, and then centrifuged for 3-5 s.

3. the test tube was subjected to ice bath, and then added with the following reagents:

| Reaction System | Volume (μL) |
|---|---|
| 5*RT Buffer | 4 |
| Thermo Scientific Ribolock Rnase Inhibitor | 0.5 |
| (20 U) | |
| RevertAid Premium Reverse Transcriptase | 1 |
| (200 U) | |

4. the test tube was gently mixed to uniform, and centrifuged for 3-5 s.

5. A reverse transcription reaction was carried out on a PCR machine under the following conditions: incubation at 25° C. for 10 min, cDNA synthesis at 50° C. for 30 min, and reaction termination at 85° C. for 5 min. The resulting cDNA product was stored at −20° C.

(4) Real-Time PCR

1. According to the gene sequence provided in the literature, the primers were synthesized by Sangon Biotech (Shanghai) Co., Ltd., and the primer sequences of β-actin, APG5L, Apg7, p62 , Bcl-1 and FASN were shown in the table below:

Sequences of Real-Time Fluorescent Quantitative PCR Primers

| Target | | |
|---|---|---|
| Gene | Primer Sequence | |
| β-actin | Forward: 5′-TAGTTGCGTTACACCCTTTCTTG-3′ | (SEQ ID NO. 1) |
| | Reverse: 5′-TCACCTTCACCGTTCCAGTTT-3′ | (SEQ ID NO. 2) |
| APG5L | Forward: 5′-GCTCTTCCTTGGAACATCACAG-3′ | (SEQ ID NO. 3) |
| | Reverse: 5′-ATCCCATCCAGAGTTGCTTGT-3′ | (SEQ ID NO. 4) |
| Apg7 | Forward: 5′-CAAGGTCAAAGGACGAAGATAAC-3′ | (SEQ ID NO. 5) |
| | Reverse: 5′-GGTCACGGAAGCAAACAACT-3′ | (SEQ ID NO. 6) |
| p62 | Forward: 5′-TAGGAACCCGCTACAAGTGC-3′ | (SEQ ID NO. 7) |
| | Reverse: 5′-ACCCGAAGTGTCCGTGTTT-3′ | (SEQ ID NO. 8) |
| Bcl-1 | Forward: 5′-GCATGTTCGTGGCCTCTAAG-3′ | (SEQ ID NO. 9) |
| | Reverse: 5′-GTTTGCGGATGATCTGTTTGT-3′ | (SEQ ID NO. 10) |
| FASN | Forward: 5′-CTTCCGAGATTCCATCCTACG-3′ | (SEQ ID NO. 11) |
| | Reverse: 5′-CAGTCAGGCTCACAAACGAAT-3′ | (SEQ ID NO. 12) |

Composition of Real-time fluorescent quantitative PCR reaction system

| Reaction System | Concentration | Volume (μL) |
|---|---|---|
| SybrGreen VCR Master Mix | 2X | 10 |
| Primer F (10 μM) | 10 μM | 0.4 |
| Primer R (10 μM) | 10 μM | 0.4 |
| ddH₂O | | 7.2 |
| Template (cDNA) | | 2 |
| Total volume | | |
| 20 μL | | |

2. The above reaction solution was placed into a real-time quantitative PCR instrument for amplification, and the PCR reaction conditions were as shown in the table below:

Real-Time Fluorescent Quantitative PCR Reaction Program

| | | | | Fluorescence |
|---|---|---|---|---|
| Program | Temperature (° C.) | Time (s) | Cycles | measure |
| Pre-denaturing | 95 | 180 | 1 | |
| Denaturing | 95 | 3 | 45 | |
| Annealing | 60 | 30 | | |
| Extending | 72 | 20 | | |
| Melting curve | 70 | | 1 | Process of |
| | | | | alleviating |
| | | | | and heating |

The fluorescence signal was collected at the end of each cycle, and finally a melting curve was drawn to determine whether the reaction product had a primer-dimer and non-specific amplification. The number of domain cycles of a fluorescence signal was recorded when the intensity of the fluorescence signal exceeded a baseline, and in the real-time quantitative PCR, a Ct value is considered to be closely related to the initial concentration of the amplified gene.

(5) Relative Quantitative Analysis

For the expression of the target gene, the difference was corrected with a reference gene β-actin by a Ct value comparison method, and the changing multiple was 2^{-ΔΔCt}. The calculation formula was: the changing multiple=2^{-ΔΔCt}, where ΔΔCt=(Ct of the target gene−Ct of the reference gene) the drug treatment group−(Ct of the target gene−Ct of the reference gene) control.

1.2.6 Statistical Analysis

Statistical analysis was conducted using the software Graphpad Prism 7.0. The comparison of mean between two groups was performed using an independent-sample t test. The comparison of mean among multiple groups was performed using one-way ANOVA, and the experiment statistical data were expressed as average ±standard deviation (x±s), where * represented that P<0.05, which indicated statistical significance, and ** represented P<0.01, which indicated that the difference was very statistically significant.

1.3 Results and Analysis

1.3.1 Effect of a Concentration of Lycium Ruthenicum Anthocyanin on the Proliferation Activity of a HepG2 Cell

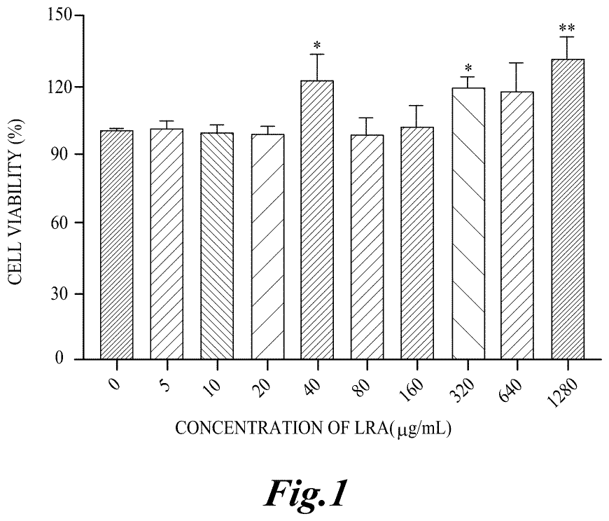

We observed the effect of Lycium Ruthenicum anthocyanin on the proliferation viability of HepG2 cells by an MTT experiment, and the results were as shown in FIG. 1. No significant decrease in the proliferation viability was seen compared with a control group when the cells were treated with Lycium Ruthenicum anthocyanin at the concentration of 0-1,280 μg/mL for 6 h, preliminarily indicating that the Lycium Ruthenicum anthocyanin was not cytotoxic within such a concentration range; when the concentration of anthocyanin was 160-1,280 μg/mL, the cell viability was significantly increased compared with the control group, indicating that the Lycium Ruthenicum anthocyanin at a higher concentration could effectively increase the cell viability and concentration in such a range could be used as the optimal experimental concentration for subsequent experiments.

1.3.2 Effect of Lycium Ruthenicum Anthocyanin on the Cellular Reactive Oxygen of HepG2 Cells

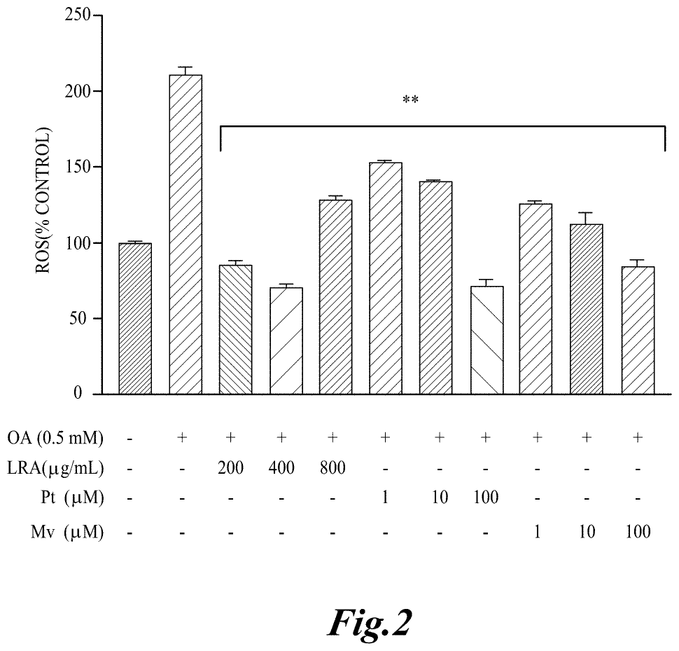

ROS is an oxygen free radical in a cell. Excessive ROS will damage mitochondria and other organelles of the cell, and thus the ATP reduction of the cell may make it impossible for the cell to continue the completion of a normal lipid metabolism reaction. We performed DCFH-DA staining on the HepG2 cells after the drug treatment, and the results were as shown in FIG. 2. The comparison between the LRA-treated group and the Control group showed that, LRA could significantly reduce the production of ROS in the cells, where the 400-800 μg/mL dosage group had the best effect, and the 1,000 μg/mL dosage group has no significant difference in effect from the 800 μg/mL dosage group.

1.3.3 Inhibition Effects of petunidin-3-O-β-D glucoside and malvidin-3-O-β-D glucoside on Cell Lipid Deposition

We also qualitatively studied the contents of TG, TC, ALT and AST in a cell of a nonalcoholic fatty liver disease model, while studying the effect of the time of treatment with petunidin-3-O-β-D glucoside and malvidin-3-O-β-D glucoside on the cells by using an MTT cell viability experiment. In the experiment, the cell treatment manner and the dosing manner were referred to 1.2.3, and the culture, model establishment and experimental steps and treatment of the cells were the same as the specific treatment methods adopted after the cell collection was completed in 1.2.4. The remaining treatment methods were as follows:

Effect of Oleic Acid on Lipid Droplet Accumulation in HepG2 Cells

A sterile circular slide were placed in a 24-well plate, added with 400 μL of DMEM, and incubated in an incubator for 30 min. The medium is pipetted off, the cells were seeded onto a glass slide at 5×10⁴ per mL, each well contained 1 mL of culture medium, the glass slide was pressed with a needle to discharge bubbles under the glass slide, the glass slide was placed into an incubator for incubation, with the culture medium being replaced every 2 days. After the cells were completely confluent, 0.5 mM oleic acid was added to treat them for 24 h. After completion of the treatment, the glass slide was taken out with a tweezer and washed for 3 times with PBS, fixed with 4% neutral formaldehyde for 30 mM, washed with PBS, then dried in the air, and added with an oil red O dye liquor which was allowed to stand on the surface of the cell for 60 mM. The excess amount of the dye liquor was pipetted off. The cells were washed with 70% ethanol, and then washed in double distilled water for 2-3 times. One drop of PBS was added dropwise on the glass slide, and then observed and photographed under a microscope.

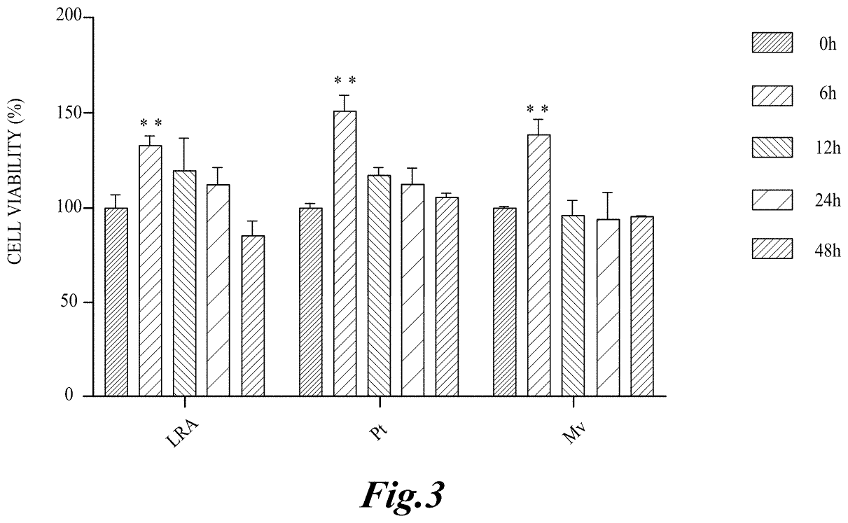

FIG. 3 showed that, the cell viability was significantly increased when the HepG2 cells had been treated with anthocyanin LRA and Pt and My for 6 h, and there was no significant difference among several other groups of treatment time, indicating that the treatment of cells with anthocyanins and petunidin-3-O-β-D glucoside, malvidin-3-O-β-D glucoside was not cytotoxic with the increase in time.

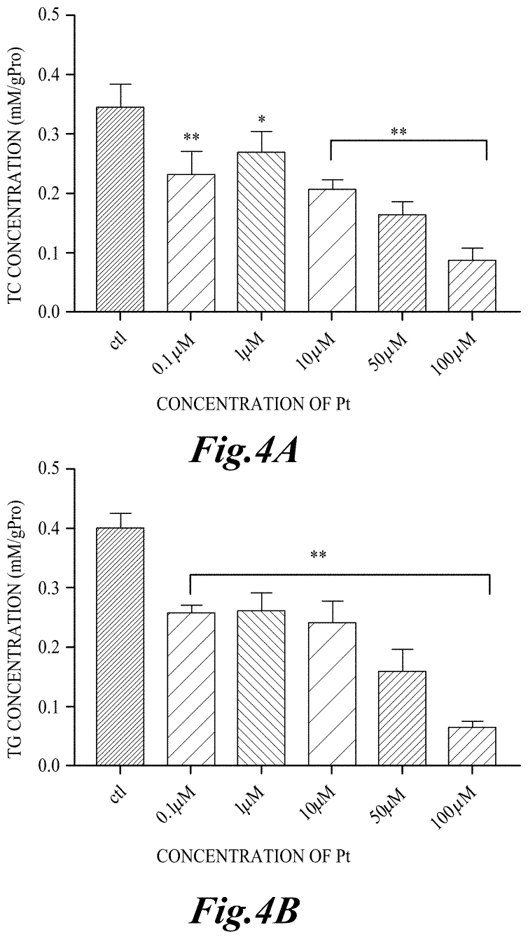

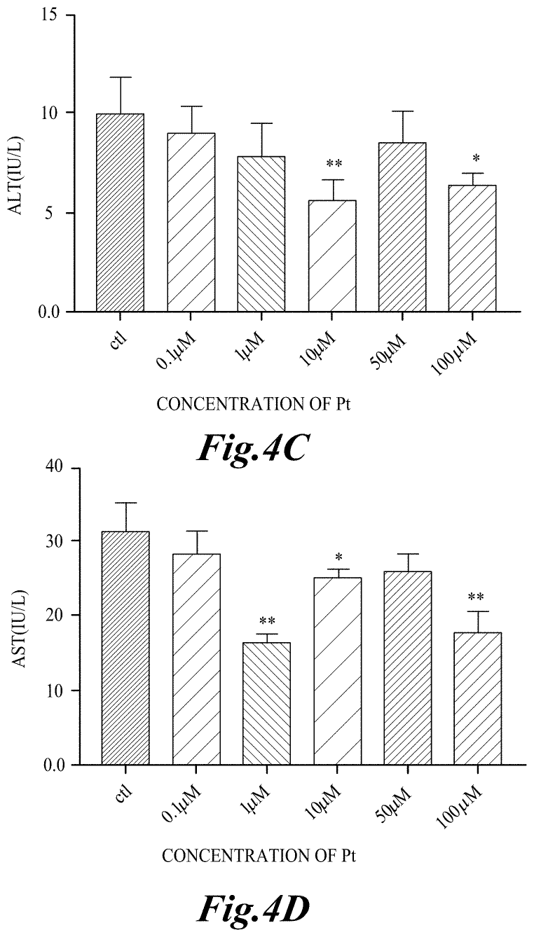

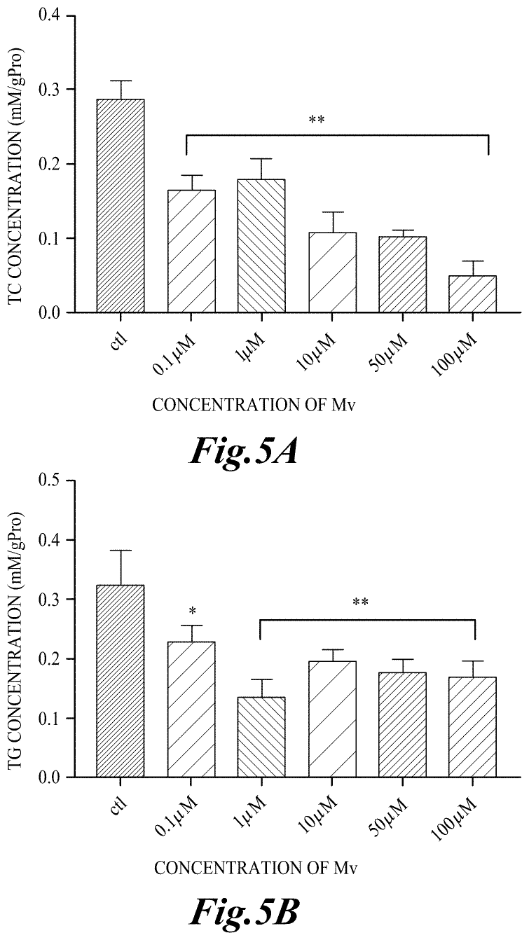

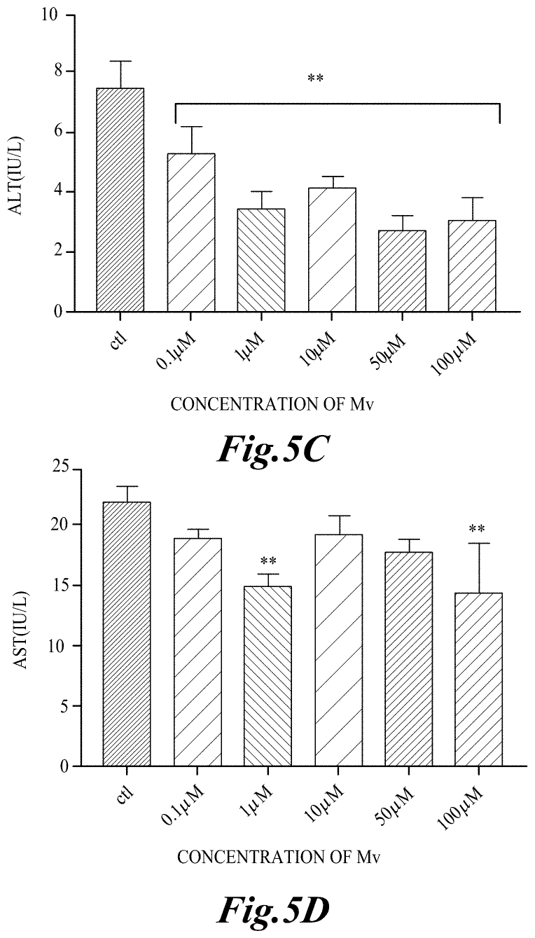

FIGS. 4A, 4B, 4C, 4D, 5A, 5B, 5C, and 5D showed that, petunidin-3-O-β-D glucoside and malvidin-3-O-β-D glucoside could reduce the levels of TC, TG, ALT and AST in a high fat cell. As compared with a control group and a low dose group, the treatment groups with medium and high doses had a very significant reduction.

1.3.4 Effect of petunidin-3-O-β-D glucoside and malvidin-3-O-β-D glucose on Autophagy of HepG2 Cells

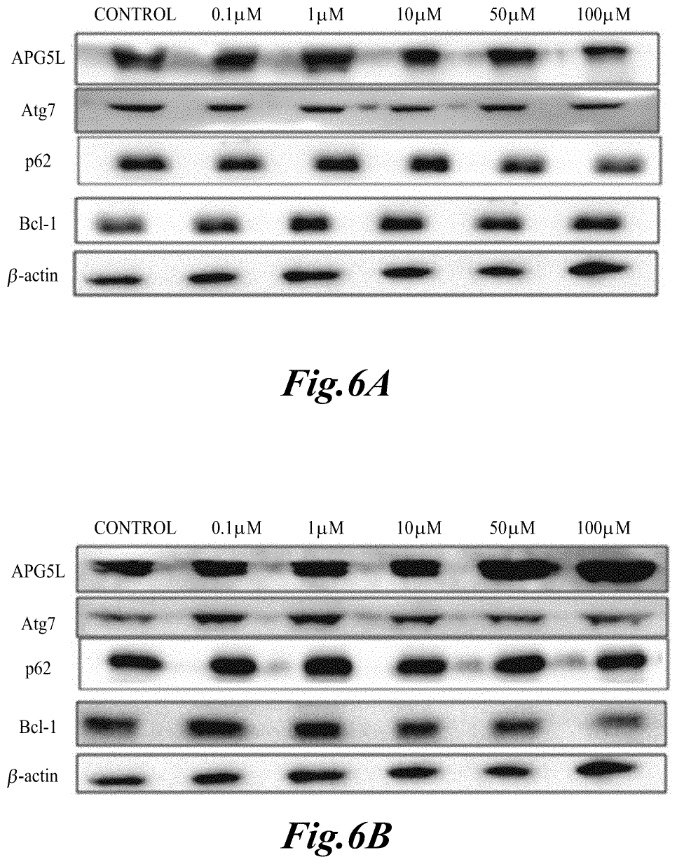

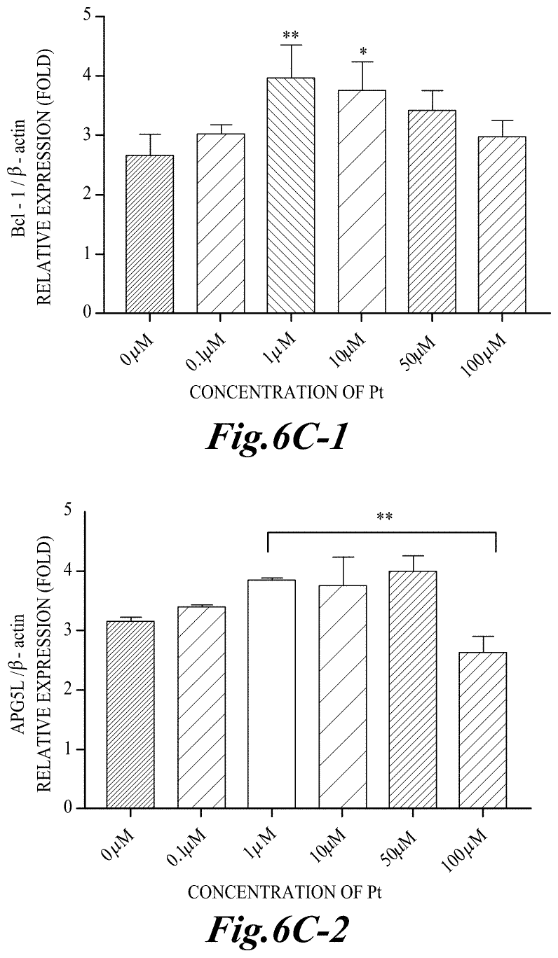

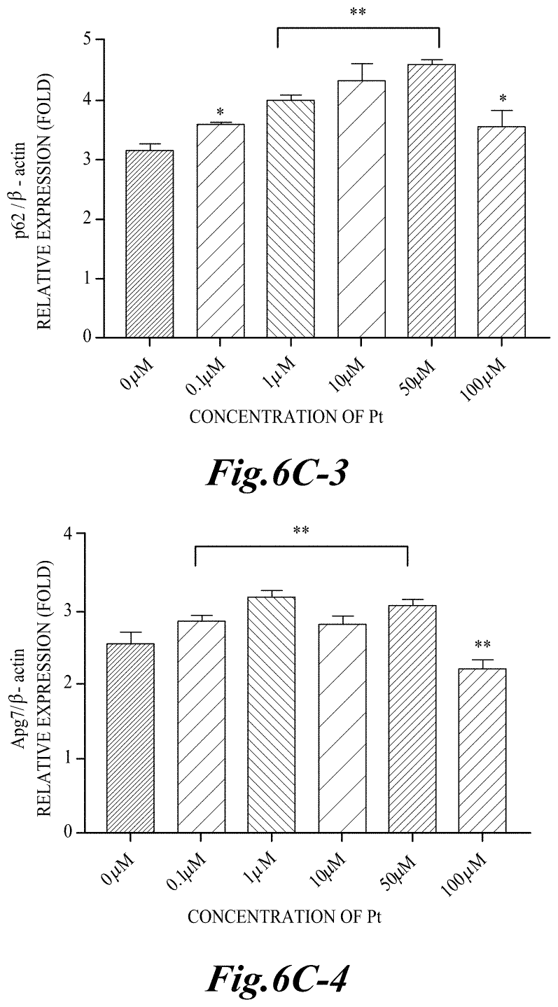

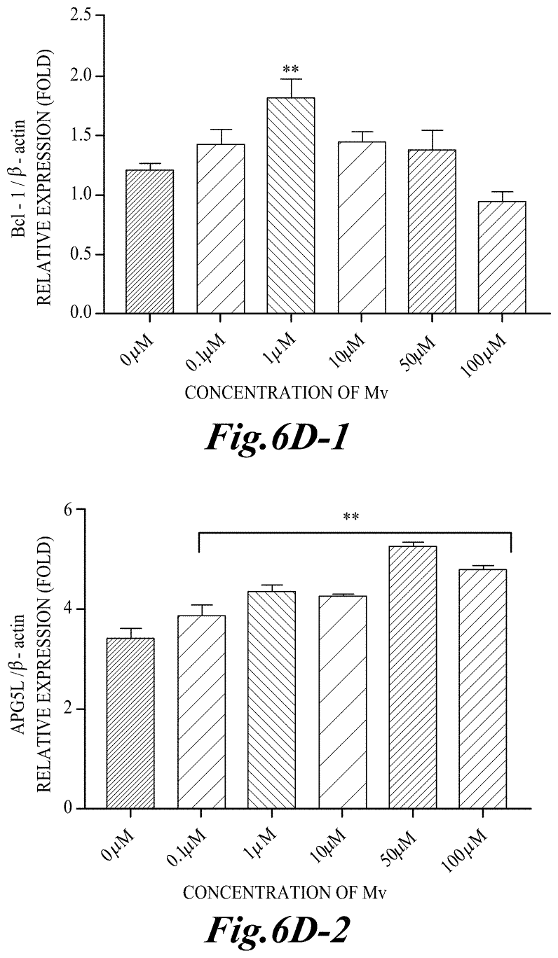

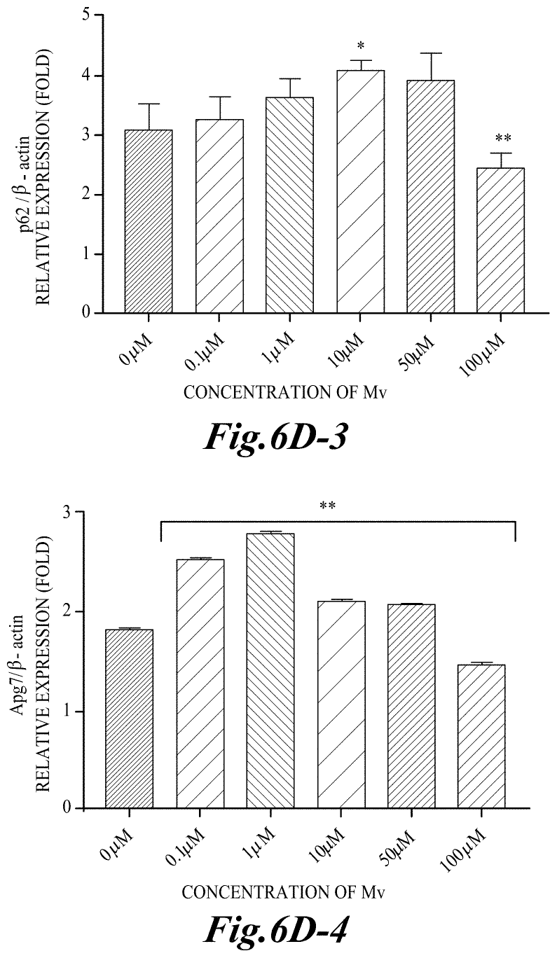

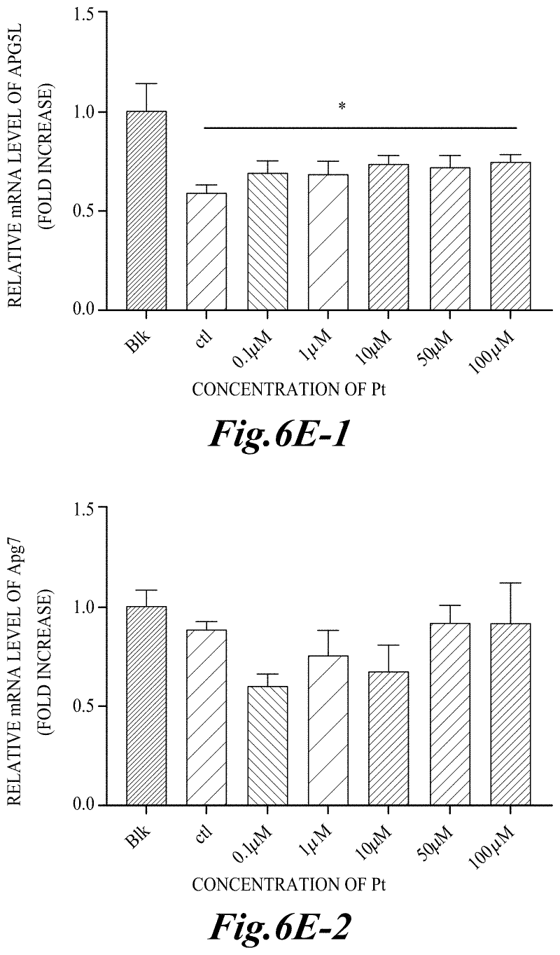

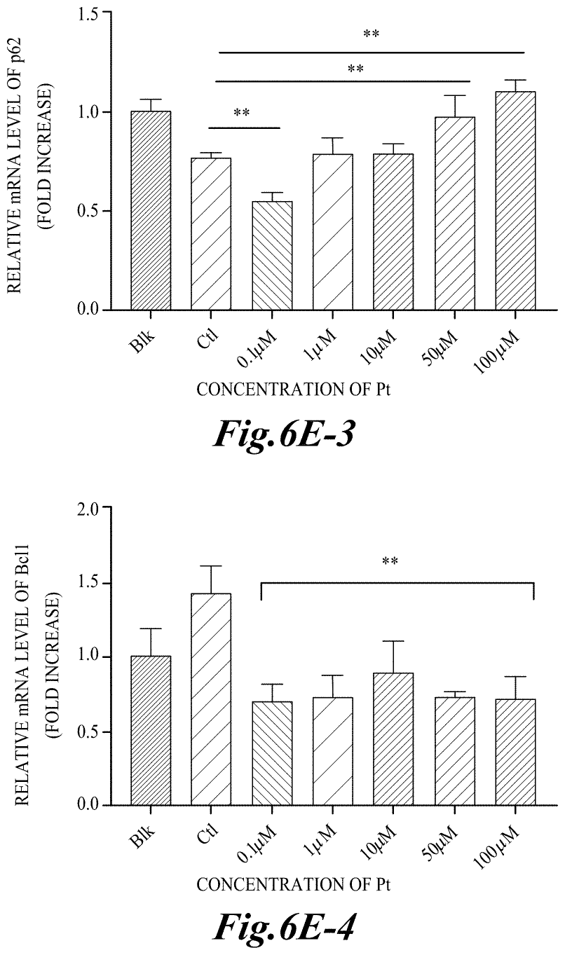

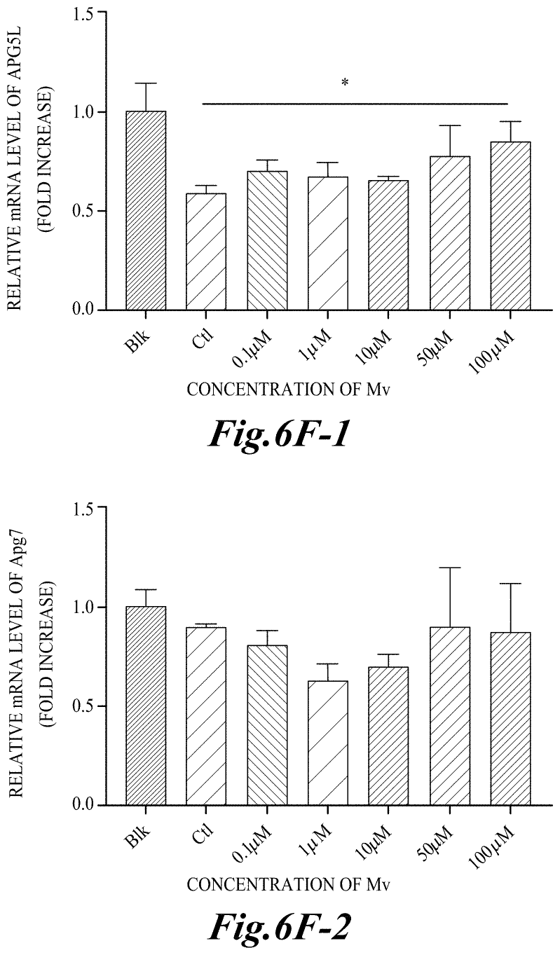

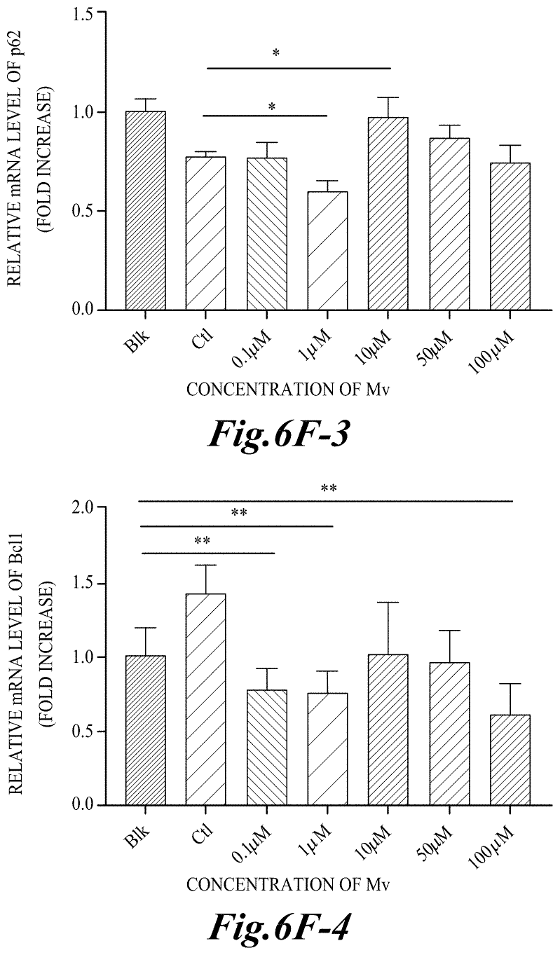

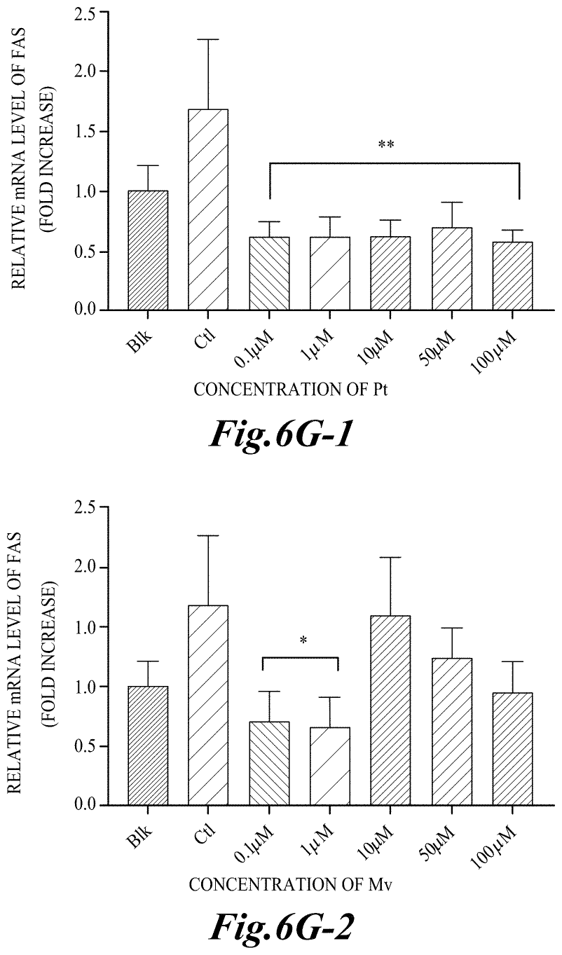

We studied the effects of petunidin-3-O-β-D glucoside and malvidin-3-O-β-D glucoside on various autophagy pathway media by Western blot and Q-PCR (FIGS. 6A to 6G-2), including Bcl-1, p62, Apg5L, APG7 and Fas, the results of Western blotting show that the low doses of petunidin-3-O-β-D glucoside and malvidin-3-O-β-D glucoside have0 a significant effect on the protein expression of APGSL, APG7, p62 and Bcl-1, The results indicated a inhibition trend in the high dose group. The propagation of LC3-II/LC3-I was relatively low, the expression of the p62 protein was relatively higher, and thus there might be activation of an autophagic flow. the results of Q-PCR showed that, petunidin-3-O-β-D glucoside and malvidin-3-O-β-D glucoside could significantly decrease the mRNA expression level of Bcl-1 and Fas, and up-regulate the level of APGSL. The low and medium doses reduced the expression of Apg7 and p62, while the high dose group could up-regulate their mRNA levels. When the doses of different groups reached 100μM, the inhibition and down-regulation of the expression levels occurred in each case.

The above description is only embodiments of the present invention and is not intended to limit the patent scope of the present invention. Any equivalent structure or equivalent flow transformation made by using the contents of the specification of the present invention, or direct or indirect uses in other related technical fields, are similarly included in the claimed patent scope of the present invention.

The embodiments of the invention in which an exclusive property or privilege is claimed are defined as follows:

1. The use of a benzopyran compound in the preparation of a product for regulating lipid metabolism, wherein the benzopyran compound comprises petunidin-3-O-β-D glucoside and/or malvidin-3-O-β-D glucoside.

2. The use according to claim 1, wherein the product is a drug used for the prevention and/or treatment of one or more of diseases associated with obesity, fatty liver, and lipid metabolism disorders.

3. The use according to claim 1, wherein the product is a lipid deposition inhibitor.

4. The use according to claim 1, wherein the product is an autophagy modifier.

5. The use according to claim 4, wherein the product is an autophagy inducing agent.

6. The use according to claim 1, wherein the product is one or more of a TC inhibitor, a TG inhibitor, an ALT inhibitor, and an AST inhibitor.

7. The use according to claim 1, wherein the product is a ROS inhibitor, and preferably a non-alcoholic fatty liver cell agonist.

8. The use according to claim 1, wherein the fatty liver is a nonalcoholic fatty liver, and the nonalcoholic fatty liver further comprises nonalcoholic simple fatty liver or/and nonalcoholic steatohepatitis.

9. The use according to claim 7, wherein the fatty liver is a nonalcoholic fatty liver, and the nonalcoholic fatty liver further comprises nonalcoholic simple fatty liver or/and nonalcoholic steatohepatitis.

10. A composition of benzopyran compounds, comprising a combination of one or more of the benzopyran compound or a stereoisomer, hydrate, solvate, pharmaceutically acceptable salt or eutectic thereof.

11. The composition according to claim 10, wherein the active ingredients of the composition comprise petunidin-3-O-β-D glucoside and/or malvidin-3-O-β-D glucoside.


SEQUENCE LISTING (12 sequences)

SEQ ID NO: 1 - DNA, 23 bp, Artificial sequence
  Synthetic
tagttgcgttacaccctttcttg

SEQ ID NO: 2 - DNA, 21 bp, Artificial sequence
  Synthetic
tcaccttcaccgttccagttt

SEQ ID NO: 3 - DNA, 22 bp, Artificial sequence
  Synthetic
gctcttccttggaacatcacag

SEQ ID NO: 4 - DNA, 21 bp, Artificial sequence
  Synthetic
atcccatccagagttgcttgt

SEQ ID NO: 5 - DNA, 23 bp, Artificial sequence
  Synthetic
caaggtcaaaggacgaagataac

SEQ ID NO: 6 - DNA, 20 bp, Artificial sequence
  Synthetic
ggtcacggaagcaaacaact

SEQ ID NO: 7 - DNA, 20 bp, Artificial sequence
  Synthetic
taggaacccgctacaagtgc

SEQ ID NO: 8 - DNA, 19 bp, Artificial sequence
  Synthetic
acccgaagtgtccgtgttt

SEQ ID NO: 9 - DNA, 20 bp, Artificial sequence
  Synthetic
gcatgttcgtggcctctaag

SEQ ID NO: 10 - DNA, 21 bp, Artificial sequence
  Synthetic
gtttgcggatgatctgtttgt

SEQ ID NO: 11 - DNA, 21 bp, Artificial sequence
  Synthetic
cttccgagattccatcctacg

SEQ ID NO: 12 - DNA, 21 bp, Artificial sequence
  Synthetic
cagtcaggctcacaaacgaat